(19) United States
(12) Patent Application Publication
(10) Pub. No.: US 2008/0200840 A1
(43) Pub. Date: Aug. 21, 2008

(54) Structural quantification of cartilage changes using statistical parametric mapping

(76) Inventors: Jose Gerarado Tamez-Pena, Pittsford, NY (US); Monica Barbu-McInnis, Alexandria, VA (US); Saara Totterman, Rochester, NY (US)

Correspondence Address: BLANK ROME LLP, 600 NEW HAMPSHIRE AVENUE, N.W., WASHINGTON, DC, 20037, US

(21) Appl. No.: 11/707,277

(22) Filed: Feb. 16, 2007

(51) Int. Cl. A61B 5/107

(52) U.S. Cl. 600/587

(57) ABSTRACT

The analysis of the focal changes in the morphology of a tissue such as cartilage is completed through statistical parametric mapping by first detecting the amount of thickness changes; followed by the point by point estimation of the variance in the thickness delta estimation. Once the change and the variance are estimated, the z-map is computed. The z-map is used to compute single change parameters. i.e: volume significant change, area of significant change, average thickness of the significant changes, and D values from the probability distributions. That can be used for treatment decisions.

### FIELD OF THE INVENTION

The present invention is directed to the evaluation of osteoarthritis progression and more particularly to such evaluation using statistical parametric mapping to evaluate cartilage degradation.

### DESCRIPTION OF RELATED ART

The detection of structural changes is an important task in the evaluation of osteoarthritis progression. The cartilage tissue is one of the primary affected structures in OA which is a debilitating disease. The early detection of OA treatment efficacy requires monitoring of small changes in cartilage morphology.

It has been reported that 70% of women and 60% of men aged 65 years or older suffer from OA. Therefore, a great deal of effort has been placed in the accurate quantification of cartilage morphological parameters such as volume, average thickness, and surface area. Cartilage tissue in normal subjects has very similar quantitative properties to OA subjects, and the evolution of those changes is very small. The problem is related to the focal nature of OA changes, which affect a small percentage of the cartilage tissue. Current approaches rely on carefully monitoring global cartilage parameters. However, they are not very sensitive to the detection of focal morphological changes in cartilage structure.

Several attempts have been proposed to address the focal nature of the disease, from direct quantification to localized analysis of sub regions of the cartilage. On the other hand, the focal nature of the OA progression has also been addressed by semi-quantitative radiological scoring, but these techniques rely heavily on human expertise. Even standard evaluation of cartilage volume depends on human intervention, which tends to introduce error in the analysis. To minimize the requirements of an expert observer for the evaluation of cartilage changes, researchers have proposed automation; even still, full cartilage analysis can not be achieved with the desired precision to detect focal changes. Therefore, new approaches are required.

In an unrelated field of endeavor, statistical parametric mapping (SPM) is a useful technique for the evaluation and localization of significant signals in medical imaging. Although SPM has been commonly used for the evaluation of activation regions in functional MRI, little effort has been made towards the application of SPM in the detection of structural changes in human anatomy.

### SUMMARY OF THE INVENTION

It is therefore an object of the present invention to present the use of SPM techniques for the detection, quantification and localization of structural cartilage changes with minimum user interaction and with just two time points.

It is another object to reduce the error in quantitative MRI measurements due to the nature of human-computer performance.

To achieve the above and other objects, in the present invention, the highly correlated nature of thickness measurements and a model behavior of random errors across areas of lower contrast are coupled to get an estimation of the thickness measurement errors. The error and the actual changes create a map of the probable location of cartilage defects that can be quantified for size. Furthermore, the approach creates probabilistic distributions of the changes that can be further explored to gain statistical insight of the changes in the cartilage tissue.

The analysis of the focal changes in cartilage morphology is completed by first detecting the amount of thickness changes, followed by the point by point estimation of the variance in the thickness delta estimation. Once the change and the variance are estimated, the z-map is computed. The change in thickness requires a baseline to follow-up registration of their 3D reconstruction of the cartilage thickness maps. The registration is achieved using the maximization of the bone-cartilage interface overlap. The change in thickness is computed for every cartilage point. The variance estimation is computed using a uniform structural kernel on the non-regular 3D surface grid. The local standard deviation of the difference (SDD) is estimated using the local variance and the global root mean square (RMS) of the variance. Finally, the z-map is computed by dividing the thickness change by the SDD. The analysis of the SDD map is quantified to provide areas of significant change, the volume of the change and the average change in thickness. Furthermore, the probabilistic distribution functions (PDF) of the thickness change are computed. The PDF is further analyzed to provide evidence of the differences in the global behavior among the different cartilage components.

Quantification of structural changes using SPM is a very useful technique for the evaluation of focal changes in cartilage morphology. The technique was evaluated with synthetic data and reduced the total system variability and improved the system ability to detect focal changes. It also provided the location and size of newly formed regions. The application of this concept in nine healthy volunteer data sets demonstrated that there is minimal change in cartilage morphology over one year. Although no major changes were observed, the delta map approach improved the precision and the ability to detect smaller changes in cartilage morphology. The improved precision was archived by the ability to map the baseline segmentation into the follow-up segmentation and look for changes on all the points that shared a common supporting area. The use of a common area reduces the errors in the definition of the extent of the cartilage surface. Furthermore, precision gains are achieved by the ability to compensate for magnet distortions. The distortions are compensated by adjusting the global cartilage shape to mimic one time point to the other time point. The ability to correct and map all the cartilage points to each other allows for the measurement of the thickness error and the estimation of areas with significant changes. The precision of those significant changes were better that the precision of the raw changes; hence the ability to detect changes is improved. On the other hand, the additional extracted information from the SPM was used to test differences among the distribution of two cartilage plates. The test in change among two distributions can be used to test the difference among two different groups, which is important when evaluating drug efficacy in newly designed disease modifying osteoarthritis drugs (DMOAD).

### BRIEF DESCRIPTION OF THE DRAWINGS

A preferred embodiment of the present invention will be set forth in detail with reference to the drawings, in which:

FIG. 1 is a flow chart showing the process according to the preferred embodiment;

FIG. 2 is a plot showing a model of the relative errors in boundary placement as a function of signal contrast;

FIGS. 3A-3F show an identification of significant changes in cartilage;

FIG. 4 shows a slice of an MRI data set with possible areas of significant loss marked;

FIGS. 5A and 5B show renderings of a femoral cartilage baseline and a statistical parametric map, respectively;

FIG. 6 is a plot showing a change in scale between baseline and follow-up scans;

FIGS. 7A-7C are plots showing relative changes of volume and thickness and the precision of the volume measurements, respectively;

FIGS. 8A-8C are plots showing the distribution of the change in thickness and its corresponding probabilistic distribution functions;

FIGS. 9A and 9B show the computation of distribution difference functions using continuous distribution functions; and

FIG. 10 is a block diagram showing a system on which the preferred embodiment can be implemented.

### DETAILED DESCRIPTION OF THE PREFERRED EMBODIMENT

A preferred embodiment of the present invention will be set forth in detail with reference to the drawings, in which like reference numerals refer to like elements or steps throughout.

The data set used for the evaluation of the method consisted of a pair of scans from nine healthy volunteers between 31 and 71 years old (mean age of 44), five female and four male, with no clinical evidence of OA. One knee of each of the nine volunteers was scanned using a GE Sigma 1.5 T scanner (GE, Milwaukee, Wis.) at two time points: baseline scan and the one year follow-up. All volunteers consented to the study protocol which was previously approved by the Institutional Research Subjects Review Board. Two sets of images were acquired for each knee: first a sagittal 3D SPGR fat saturated sequence with a TR of 39, TE of 7.0 ms, Nex 1, Flip angle of 20°, and a 256 by 256 matrix with a 14.0 cm field of view. Second, a sagittal 3D GRE image with a TR of 29, TE of 15.0 sec, Nex 1, Flip angle of 40°, and a 256 by 256 matrix with a 14.0 cm field of view were acquired.

The extraction of the cartilage tissue followed the methods described in Tamez-Peña J, Barbu-McInnis M, Totterman S, “Knee Cartilage Extraction and Bone-Cartilage Interface Analysis from 3D MRI Data Sets”, SPIE Medical Imaging, 5370, 1774-1784, 2004, and in Tamez-Peña J, Barbu-McInnis M, Totterman S “Unsupervised Definition of the Tibia-Femoral Joint Regions of the Human Knee and its Applications to Cartilage Analysis”, SPIE Medical Imaging, 6144, 2006. The extraction of cartilage tissue was 3D rendered using an enhanced 3D reconstruction algorithm that was able to interpolate the surface boundary in areas with no-contrast. The 3D reconstruction also included the corresponding labels of the cartilage regions. Once the cartilage was 3D rendered, the thickness computation was done using a heuristic approach that combined the normal thickness computations described in the 2006 paper just cited and the thickness computation of the minimum point-to-point distance between the articular surface and the subchondrial bone plate. The combination of the two methods allowed the determination of thickness values at regions with cartilage defects and under the presence of bone osteophytes.

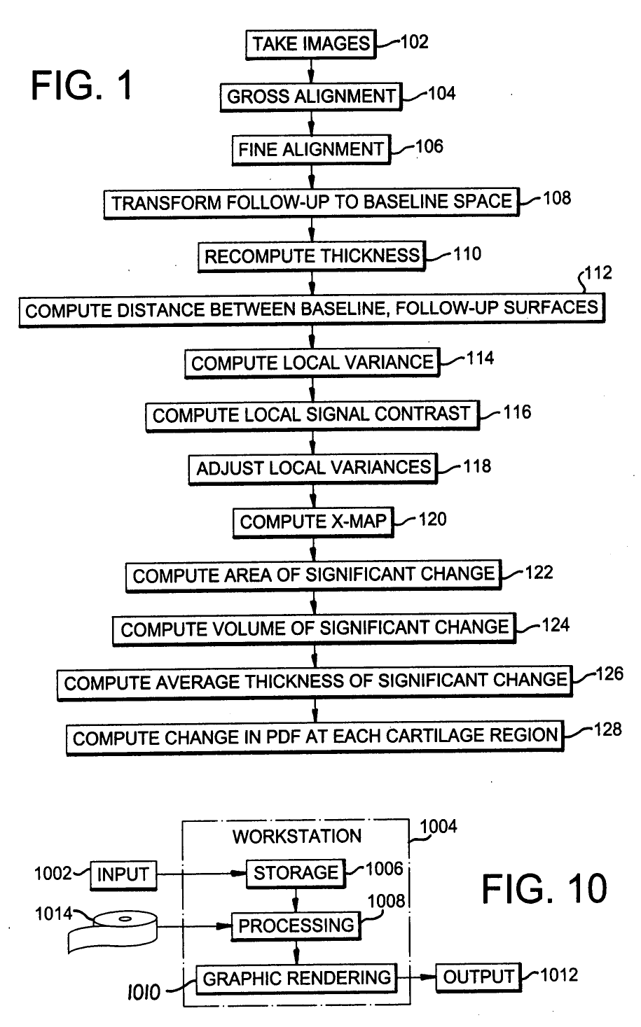

Once the cartilage was 3D rendered at the two time points by the enhanced approach, the next step was the computation of the significant changes. The following steps describe the approach, as shown in the flow chart of FIG. 1:

In step 102, the images are taken. Then the subchondrial bone plate areas are registered in the following fashion:

Step 104: Gross alignment of the areas by computing the inverse rotation matrix using the scattering matrix that is defined by all the cartilage points that are in contact with the bone.

Step 106: Fine alignment of the subchondrial bone plates by maximizing the overlapping area. The overlapping maximization includes nine degrees of freedom: three for translation, three for rotation, and three for scale (only applied for the fitting of for the femoral cartilage). The maximization is completed using a greedy maximization approach in a multi-resolution scheme.

Step 108: Transform the follow-up rendering into the baseline space.

Step 110: Re-compute the follow-up cartilage thickness in the baseline space.

Step 112: For each point in the baseline surface: compute the point to point distance between the baseline surface and the follow-up surface.

Step 114: Compute the local variance of thickness difference.

Step 116: Compute the local signal contrast between the cartilage surface and the adjacent tissues.

Step 118: Adjust the local variances using the pooled estimation:

Var=(Local Var+Voxle Res*M(c))*0.5

where c is the contrast and M(c) is the correction factor associated with each contrast value.

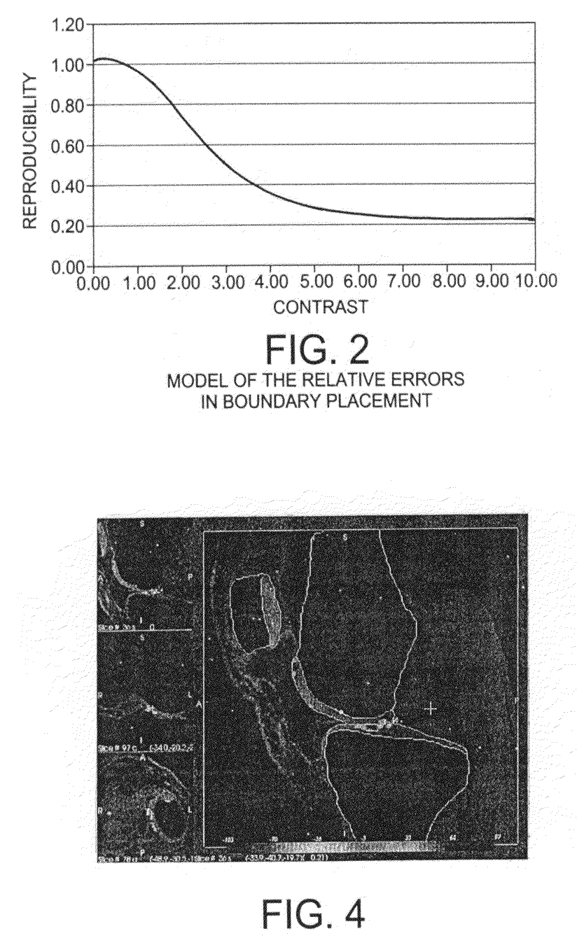

Step 120: Compute the z-map: The measured change in thickness divided by the local pooled estimation of the standard deviation of the difference (SDD). SDD=(Var)^{0.5}. FIG. 2 shows a model of the SD behavior as a function of the signal contrast. The variability (random error) in locating the right location of the cartilage boundary depends on the signal contrast of the cartilage to the adjacent tissues. The lower the contrast, the higher are the chances in making errors in the localization of the cartilage boundary, as described in Tamez-Peña J, Barbu-McInnis M, Totterman S, “Knee Cartilage Extraction and Bone-Cartilage Interface Analysis from 3D MRI Data Sets”, SPIE Medical Imaging, 5370, 1774-1784, 2004.

Step 122: Compute the area of significant change: Add all the surface area of all the surface faces whose change in thickness is greater than three SDD:

Area Loss: Changes lower than three SDD;

Area Gain: Changes greater than three SDD.

Step 124: Compute the volume of significant change: Add the volume of the associated change: face area times the average change observed in that face:

Volume Loss: Changes lower than three SDD;

Volume Gain: Changes greater than three SDD.

In the above steps, three SDD is given as an illustrative rather than limiting value. Other values can be used instead, e.g., 2.5 or 3.5.

Step 126: Compute the average thickness of the significant change:

Thickness Loss: Volume Loss/Area Loss;

Thickness Gain: Volume Gain/Area Gain.

Step 128: Compute the change PDF (PDF of the thickness change) at each cartilage region: For each z value of the points of the cartilage, add the total area associated to that z value and divide it by the total area of the cartilage region.

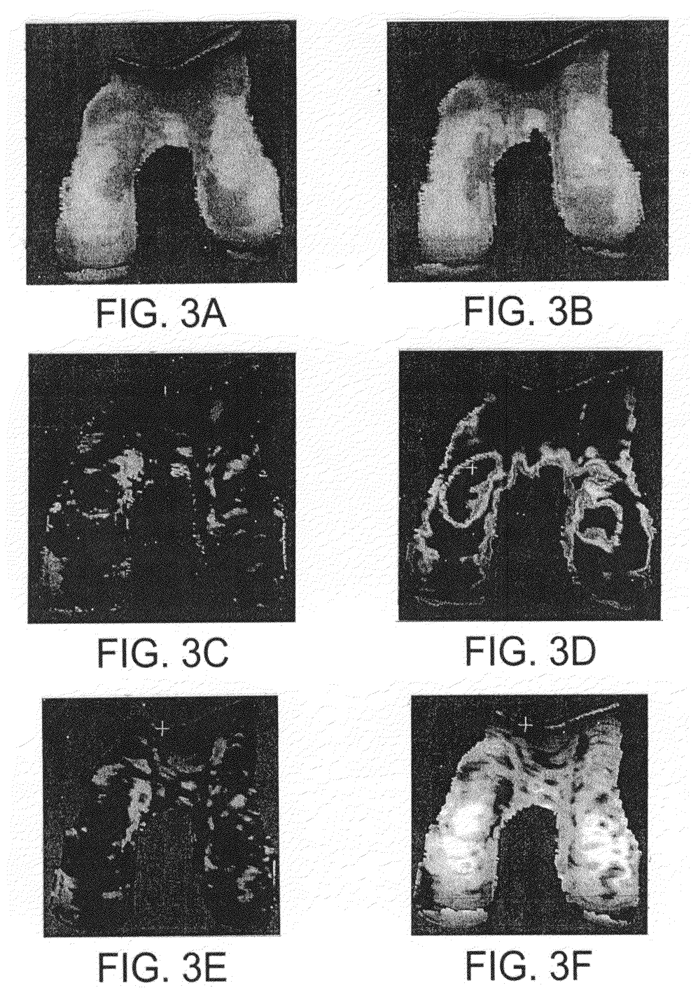

An example of an identification of the significant changes is shown in FIGS. 3A-3F. The baseline thickness map and the follow-up thickness map, shown in FIGS. 3A and 3B respectively, are registered, and a difference map is created, as shown in FIG. 3C. The difference map is used to compute the signal contrast and the local variability of the paired measurements. The contrast and the variability are used to compute the SDD map, shown in FIG. 3D. The SDD map and the thickness changes are used to compute the z-map, shown in FIG. 3E, which is then data-mined to compute the area and the volume of the significant changes, as shown in FIG. 3F.

The advantage of this approach is that significant changes can be mapped back to the MRI image and that the computer can highlight those changes. That can be seen from FIG. 4, which shows a slice of the MRI data set with possible areas of significant loss marked.

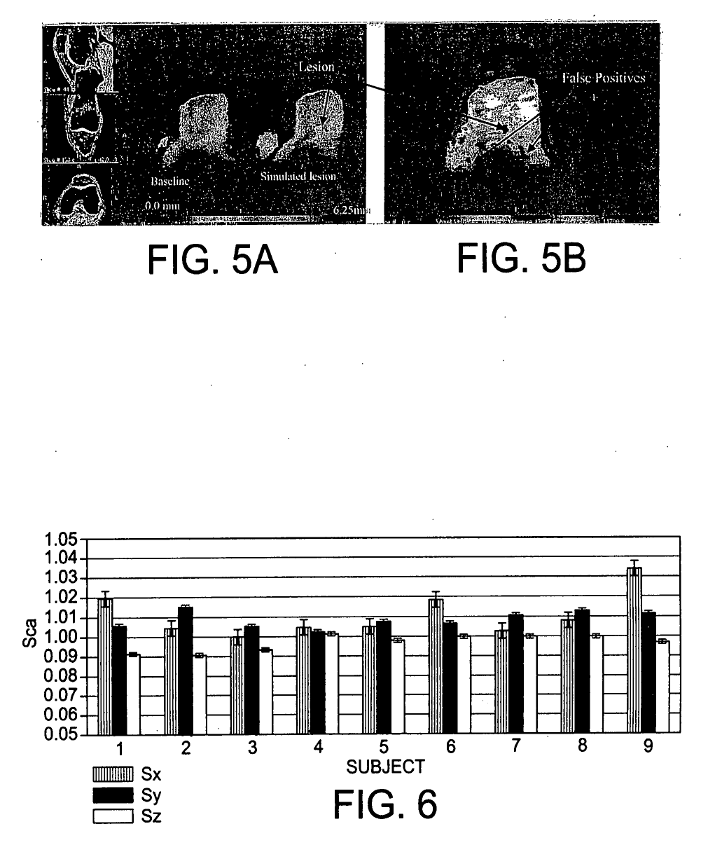

The approach described here was first tested in a controlled environment. FIGS. 5A and 5B show the 3D renderings of a single time point analysis for a subject with a simulated lesion. FIG. 5A shows the 3D rendering of the femoral cartridge baseline and the cartilage with the simulated lesion. FIG. 5B shows the SPM of the detected changes. The location of the lesion is indicated. To simulate the lesion two independent segmentation of the knee cartilage were created. Once the segmentations were completed, the second one was doctored by an expert user that created a virtual lesion. The doctored segmentation and the original segmentation were compared to the independent segmentation using the SMP approach.

Table 1 describes the results of the quantification. The analysis reanalysis resulted in some false positive identification; however, the size of those identifications was smaller than the expected size (0.5%). Once the SPM approach was complete on the doctored data set, the size of the significant change was greater than the 0.5%.

TABLE 1
Simulated Results

| | | | | Volume of | Area of | |
|---|---|---|---|---|---|---|
| | | SPM | RMS | Significant | Significant | Greater |
| | Raw | Detected | SDD | Change | Change | Than |
| | Change | Change | (mm) | (mm³) | (mm²) | Chance |
| Analysis- | −1.5% | 0.5% | 0.21 | −17.4 | 6.4 | No |
| Reanalysis | | | | | | |
| Simulated | −1.8% | 0.2% | 0.21 | −30.0 | 19.2 | Yes |
| Lesion | | | | | | |

After the successful competition of the test case we preceded to the computation of the delta maps on the nine healthy volunteers. The registration of the nine subjects created delta maps that were scaled from the independent analysis of the follow-up. FIG. 6 shows the scaled values applied to the 3D segmentations. As we can see, some significant changes in scale occurred between the baseline scan and the follow-up. The effect of the scale was most of the time greater than one. Therefore, the uncorrected data will produce artificial change in cartilage volumes.

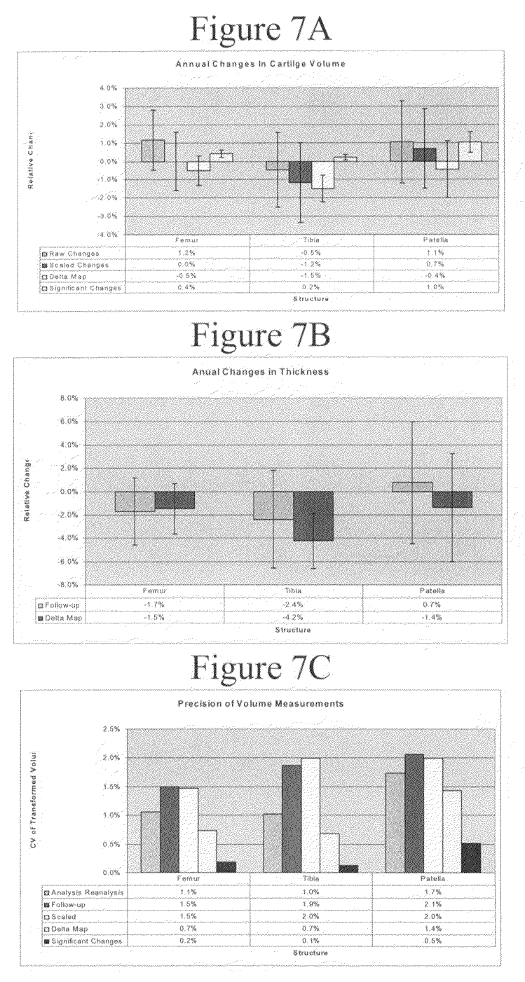

Table 2 summarizes the results of the global parameters. The volume measurements were transformed by the cubic root in order to remove error dependency due to size. The table shows that the precision of the volume measurements were improved by the delta map approach while the precision thickness measurements were not affected significantly. On the other hand, the use of the SPM affected the global measurements in a positive way as seen in FIGS. 7A, 7C and 7C, which show, respectively, the changes of volume and thickness and the relative precision of annual based volume measurements.

Table 3 and Table 4 show the amount of significant changes. As expected the amount of significant changes was very small. None of the volunteers showed clinical OA, and the MRI findings did not show any evidence of OA findings or OA progression. The only cartilage region that showed some relevant changes was the patella, which showed an increase of cartilage volume and some changes in a significant portion of the cartilage area.

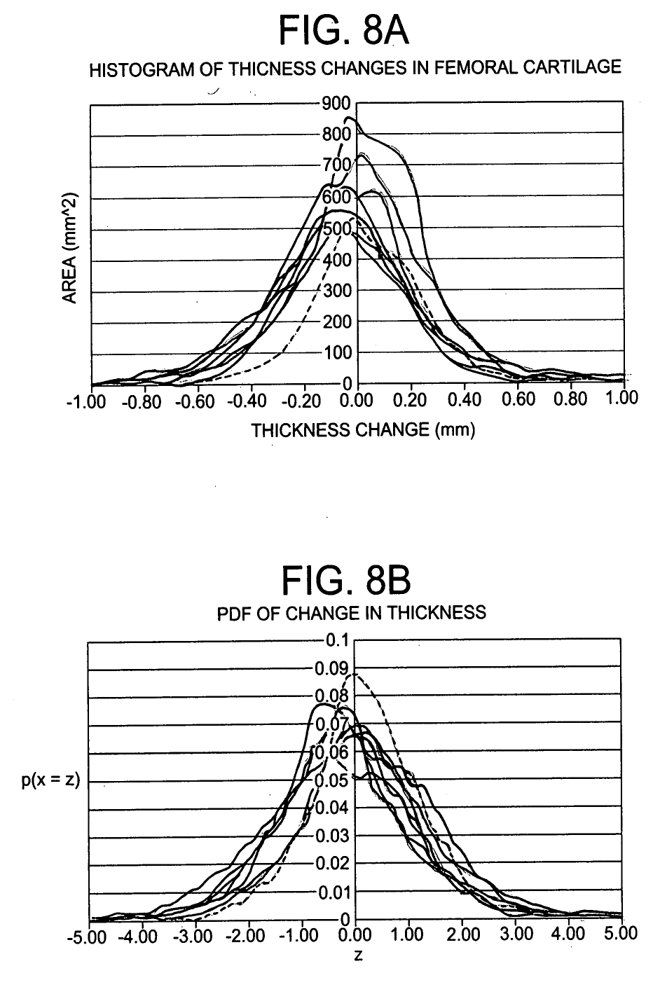

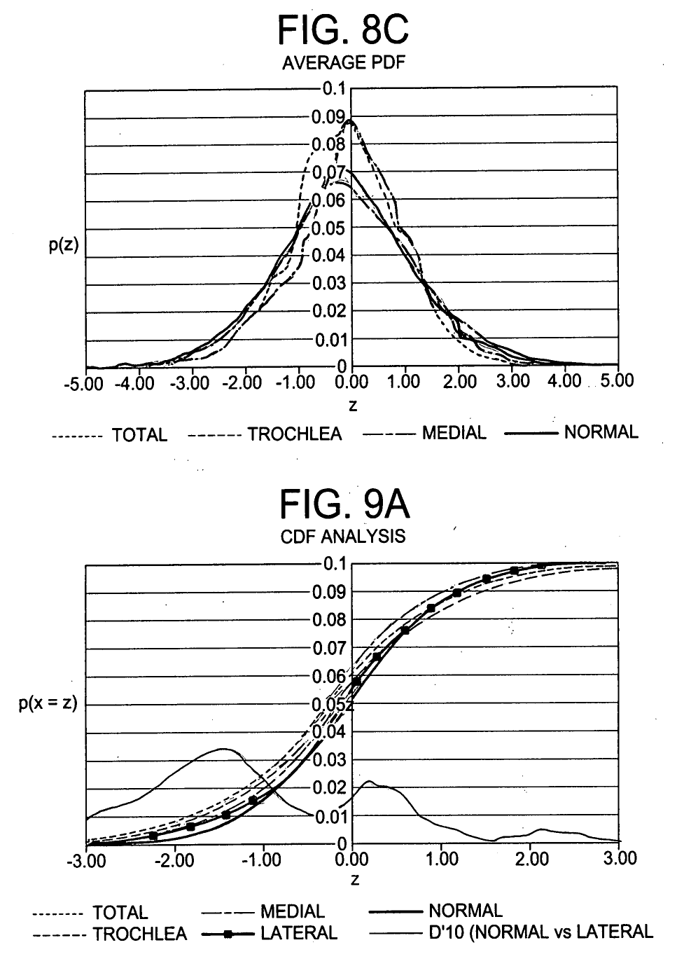

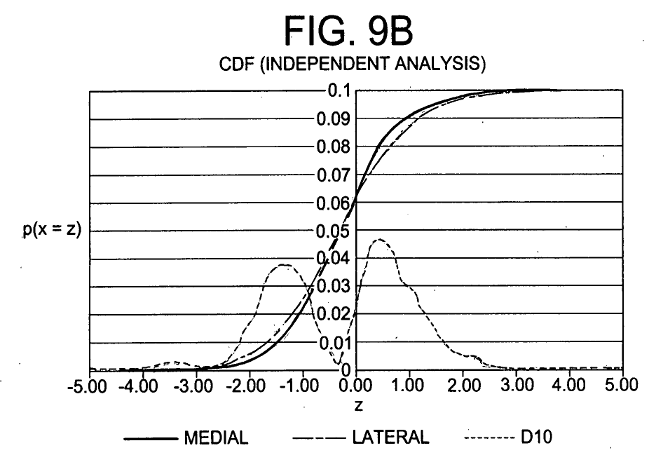

We also explored the PDF information that was created by the SPM. FIGS. 8A-8C and 9A-9B show the results. FIGS. 8A-8C show the distribution of the change in thickness and its corresponding probabilistic distributions functions (PDF). More specifically, they show, respectively, the femoral histograms of change from the nine subjects in the study, the conversion of the histograms to a PDF using the SPM data, and the population-wise regional PDF extraction from the SPM data. The PDFs were computed from the SPM maps. Because the PDFs are normalized they are less sensitive to subject size.

FIGS. 9A and 9B show that the PDF information can be used to compute distribution differences among populations using the associated continuous distributions functions (CDF). In FIG. 9A, the different behavior of the cartilage plates and the comparison of the lateral change and the Normal distribution are plotted. In FIG. 9B, the different behaviors of the medial and lateral tibia cartilages are plotted. These plots show indications of behavioral differences among different plates and deviations from the normal distributions; however, the study's small sample size can not be used to make any population generalizations.

FIGS. 8A-8C show how the histogram distribution of the thickness changes is improved by the PDF normalization. The spread and the range of the changes are more uniform in the transformed PDF space. This resulted in a tighter distribution of the PDF lines compared to the histogram lines. Once we compute the average histogram by cartilage plate we can see some differences in the distributions. The statistical evaluation of those differences in accomplished using the cumulative distribution functions (CDF). FIG. 9A shows those CDF of the major cartilage plates, while FIG. 9B compares the CDF of the medial and the lateral tibia cartilage. The computation of the D statistics (D=|p₁(x<z)−p₁(x<z)|) allows the evaluation the degree of statistical deviation from the two distributions. This parameter shows that there is some differences in progression between the Medial and Lateral side of the tibia cartilage; but this difference can be due to random occurrence of the small population.

TABLE 2
Summary of the baseline data and the measured changes in the femur, tibia
and patella cartilage
Summary Statistics of Annual Changes in Cartilage Morphology as Measured by MRI

| | | Volume^{(1/3)} (mm) | | | | | | | |
|---|---|---|---|---|---|---|---|---|---|
| | | | Raw | | | | | | |
| | | | Changes | | | | Thickness (mm) | | |
| | | | Baseline | | | | | Changes | |
| | | | vs. | Delta Map Changes | | | | | Change: |
| | | Baseline | Follow- | | Delta | Significant | Baseline | | Delta |
| | | Data | up | Scaled | Map | Changes | Thickness | Raw | Map |
| Femur | AVG | 21.93 | 0.25 | 0.00 | −0.11 | 0.09 | 2.16 | −0.04 | −0.03 |
| | STD | 2.60 | 0.46 | 0.46 | 0.23 | 0.06 | 0.36 | 0.08 | 0.06 |
| Tibia | AVG | 16.79 | −0.08 | −0.20 | −0.25 | 0.04 | 2.18 | −0.05 | −0.09 |
| | STD | 1.89 | 0.44 | 0.47 | 0.16 | 0.03 | 0.38 | 0.12 | 0.07 |
| Patella | AVG | 14.74 | 0.16 | 0.10 | −0.06 | 0.15 | 2.74 | 0.02 | −0.04 |
| | STD | 1.59 | 0.43 | 0.42 | 0.30 | 0.11 | 0.43 | 0.19 | 0.17 |

TABLE 3
Amount of Significant Changes in the Cartilage Tissue

| | Significant Changes (Absolute) | | | | | | | |
|---|---|---|---|---|---|---|---|---|
| | | | | | | | Depth of | |
| | Volume (mm³) | | | Area (mm²) | | | Changes (mm) | |
| | Gain | Loss | Total | Gain | Loss | Total | Gain | Loss |
| Femur | 19.67 | 25.27 | 44.94 | 29.67 | 39.08 | 68.75 | 0.66 | 0.60 |
| Tibia | 4.81 | 8.93 | 13.74 | 8.09 | 9.97 | 18.06 | 0.57 | 0.76 |
| Patella | 49.18 | 46.28 | 95.46 | 64.07 | 59.05 | 123.12 | 0.76 | 0.74 |

TABLE 4
Relative annual changes in the cartilage parameters

| | Significant Changes (Relative) | | | | | | | |
|---|---|---|---|---|---|---|---|---|
| | | | | | | | Relative Depth of | |
| | Volume | | | Area | | | Changes | |
| | Gain | Loss | Total | Gain | Loss | Total | Gain | Loss |
| Femur | 0.4% | 0.5% | 0.9% | 1.4% | 1.8% | 3.2% | 30.9% | 28.2% |
| Tibia | 0.2% | 0.3% | 0.5% | 0.7% | 0.8% | 1.5% | 24.8% | 33.0% |
| Patella | 1.3% | 1.2% | 2.5% | 4.9% | 4.5% | 9.3% | 27.0% | 26.1% |

The embodiment disclosed above and other embodiments can be implemented in a system such as that shown in the block diagram of FIG. 10. System 1000 includes an input device 1002 for input of the image data and the like. The information input through the input device 1002 is received in the workstation 1004, which has a storage device 1006 such as a hard drive, a processing unit 1008 for performing the processing disclosed above to provide the data, and a graphics rendering engine 1010 for preparing the data for viewing, e.g., by surface rendering. An output device 1012 can include a monitor for viewing the images rendered by the rendering engine 1010, a further storage device such as a video recorder for recording the images, or both. The software used to implement the invention can be provided on any suitable medium 1014. Illustrative examples of the workstation 1004 and the graphics rendering engine 1010 are Intel x86 systems and Open GL.

While a preferred embodiment of the present invention has been set forth above, those skilled in the art who have reviewed the present disclosure will readily appreciate that other embodiments can be realized within the scope of the invention. For example, numerical values are illustrative rather than limiting, as are specific imaging modalities. Also, the invention can be used with any joints for which it is suitable, for human and non-human animal patients. Moreover, the concept can be applied in neurological, cardiovascular, cancer and other applications as well. Therefore, the present invention should be construed as limited only by the appended claims.

We claim:

1. A method for analyzing changes in morphology of a tissue in a region of interest in a body, the method comprising:
(a) receiving image data representing first and second images of the region of interest, the first and second images being separated in time;
(b) registering the second image to the first image;
(c) from the first and second images registered in step (b), detecting a change in thickness of the tissue;
(d) from the change in thickness detected in step (c), estimating a variance in the change in thickness; and
(e) from the variance estimated in step (d), computing a map representing the variance.

2. The method of claim 1, wherein the tissue comprises cartilage.

3. The method of claim 2, wherein step (b) comprises performing a gross alignment by using an inverse rotation matrix.

4. The method of claim 3, wherein step (b) further comprises performing a fine alignment by maximizing a bone-cartilage interface overlap.

5. The method of claim 1, wherein step (c) is performed point by point over the tissue.

6. The method of claim 5, wherein step (d) is also performed point by point over the tissue.

7. The method of claim 6, wherein step (e) comprises:
(i) computing a local signal contrast between the tissue and adjacent tissues; and
(ii) adjusting the variance in accordance with a correction factor derived from the local signal contrast.

8. The method of claim 7, wherein step (e) further comprises:
(iii) dividing the change in thickness by a local pooled estimation of a standard deviation, the local pooled estimation being a function of the variance adjusted in step (e) (ii).

9. The method of claim 8, further comprising (f) computing at least one of an area, volume and average thickness of a significant change from the map.

10. The method of claim 9, further comprising computing a probabilistic distribution function of the change at each region of the tissue.

11. A system for analyzing changes in morphology of a tissue in a region of interest in a body, the system comprising:
an input for receiving image data representing first and second images of the region of interest, the first and second images being separated in time;
a processor for: registering the second image to the first image; from the registered first and second images, detecting a change in thickness of the tissue; from the change in thickness, estimating a variance in the change in thickness; and from the variance, computing a map representing the variance; and
an output for outputting the map.

12. The system of claim 11, wherein the tissue comprises cartilage, and wherein the processor is programmed to operate with regard to the cartilage.

13. The system of claim 12, wherein the processor performs a gross alignment by using a inverse rotation matrix.

14. The system of claim 13, wherein the processor also performs a fine alignment by maximizing a bone-cartilage interface overlap.

15. The system of claim 11, wherein the processor detects the change in thickness point by point over the tissue.

16. The system of claim 15, wherein the processor estimates the variance point by point over the tissue.

17. The system of claim 16, wherein the processor refines the variance map by:
(i) computing a local signal contrast between the tissue and adjacent tissues; and
(ii) adjusting the variance in accordance with a correction factor derived from the local signal contrast.

18. The system of claim 17, wherein the processor computes the map further by:
(iii) dividing the change in thickness by a local pooled estimation of a standard deviation, the local pooled estimation being a function of the adjusted variance.

19. The system of claim 18, wherein the processor further computes at least one of an area, volume and average thickness of a significant change from the map.

20. The system of claim 19, wherein the processor computes a probabilistic distribution function of the change at each region of the tissue.

21. An article of manufacture for analyzing changes in morphology of a tissue in a region of interest in a body, the article of manufacture comprising:
a computer-readable storage medium; and
code for controlling a processor for:
(a) taking first and second images of the region of interest, the first and second images being separated in time;
(b) registering the second image to the first image;
(c) from the first and second images registered in step (b), detecting a change in thickness of the tissue;
(d) from the change in thickness detected in step (c), estimating a variance in the change in thickness; and
(e) from the variance estimated in step (d), computing a map representing the variance.

22. The article of manufacture of claim 21, wherein the tissue comprises cartilage, and wherein the code is adapted for use with the cartilage.

23. The article of manufacture of claim 22, wherein step (b) comprises performing a gross alignment by using a inverse rotation matrix.

24. The article of manufacture of claim 23, wherein step (b) further comprises performing a fine alignment by maximizing a bone-cartilage interface overlap.

25. The article of manufacture of claim 21, wherein step (c) is performed point by point over the tissue.

26. The article of manufacture of claim 25, wherein step (d) is also performed point by point over the tissue.

27. The article of manufacture of claim 26, wherein step (e) comprises:
(i) computing a local signal contrast between the tissue and adjacent tissues; and
(ii) adjusting the variance in accordance with a correction factor derived from the local signal contrast.

28. The article of manufacture of claim 7, wherein step (e) further comprises:
(iii) dividing the change in thickness by a local pooled estimation of a standard deviation, the local pooled estimation being a function of the variance adjusted in step (e) (ii).

29. The article of manufacture of claim 28, wherein the code further comprises code for (f) computing at least one of an area, volume and average thickness of a significant change from the map.

30. The article of manufacture of claim 29, wherein the code further comprise code for computing a probabilistic distribution function of the change at each region of the tissue.
